(19) United States
(12) Patent Application Publication
(10) Pub. No.: US 2011/0094912 A1
(43) Pub. Date: Apr. 28, 2011

(54) Package with Divider to Separate Contents, and Method of Filing the Package at High Speed

(75) Inventors: Wayne Rossi, Dover, DE (US); Michael Williamson, Frederica, DE (US); Peter Preisner, Quinton, VA (US); Wojtek Drewnowski, Richmond, VA (US); John Stanley, Richmond, VA (US)

(73) Assignee: PLAYTEX PRODUCTS, LLC

(21) Appl. No.: 12/606,002

(22) Filed: Oct. 26, 2009

(51) Int. Cl. A61B 19/02; A61F 13/20; B65B 11/06

(52) U.S. Cl. 206/438; 53/461

(57) ABSTRACT

The present disclosure provides a divider for packaging applications. The divider can separate products in the package into a plurality of groups, and can also contain relevant information about the products, as is required in the case of packaging tampons. The present disclosure also provides a method of packaging a plurality of products, using the divider.

### BACKGROUND OF THE DISCLOSURE

1. Field of the Disclosure

The present disclosure relates to a divider for a package. In particular, the present disclosure relates to a divider for a tampon package that also includes warnings safety information relating to the tampons, and is included as part of the package. The present disclosure further provides a method of filling the package at a fast, or high speed.

2. Discussion of the Related Art

The United States Food and Drug Administration (FDA) requires certain devices to have “specific labeling, which may include not only package labeling, but informational literature, patient release forms, performance testing, and/or specific tolerances or prohibitions on labeling, but informational literature, patient release forms, performance testing, and/or specific tolerances or prohibitions on certain ingredients.” (http://www.fda.gov/MedicalDevices/DeviceRegulationandGuidance/Overview/DeviceLabeling/GeneralDeviceLabelingRequirements/ucm052182.htm, last visited Oct. 19, 2009)

There are special labeling requirements for certain devices, one of which is menstrual tampons. The labeling requirements for tampons are mostly directed to relevant information about Toxic Shock Syndrome (TSS). 21 C.F.R. §801.430.

Tampons are often sold in boxes of a dozen or more. For years, all commercial tampon manufacturers have included paper inserts, specifically information booklets, in boxes of tampons, which communicate both safety and recommended usage information pertaining to menstrual tampons. (See FIG. 1) The inserts are usually wrapped around a group of tampons during the packaging operation, and the group of tampons are “pushed” into the insert during assembly of the package.

This traditional method of conveying information using a paper insert/information booklet is both uneconomical and operationally inefficient, as the packaging machines will often jam. The paper used for inserts is thin, and folded in several places, so including the insert in packages of tampons produced at high speeds involves mechanical and operational complexity. Conditions as commonplace as high heat or humidity, or even minor folds or tears in the paper, can cause errors in the packaging process, which lead to frequent machine shutdowns and product rejections. This process thus involves unnecessary cost, scrap, and waste. Accordingly, there is a need to overcome these disadvantages. There is also a need to fill such a package, or a number of such packages, at a high speed.

### SUMMARY OF THE DISCLOSURE

The present disclosure provides a way of communicating safety and usage information for medical devices, in particular TSS information pertaining to menstrual tampons, which avoids the above-discussed disadvantages of currently available methods. The present disclosure provides a special “divider” that is a part of the package containing individually wrapped tampons. The divider contains essentially the same safety and recommended usage information as that provided in the more traditional package insert/information booklet. Since the divider would be part of the actual package containing the individually wrapped tampons, inclusion of the divider would be trivial to the packaging operation. There would be no loss of information, lower mechanical complexity, less labor, and lower scrap. Also, due to the features of the process of filling the package with the divider, more packages are filled per unit time.

Thus, in one embodiment, the present disclosure provides a package having a package body having an opposable top, a bottom, and a wall, to define an interior space, the interior space for receipt of a plurality of products, and a divider in the interior space, wherein the divider separates the products into at least two separate groups of products, and wherein the divider has information relating to the products printed thereon, the information being displayed prominently when the package body is opened

In another embodiment, the present disclosure provides A method of filling a package with a plurality of products. The method comprises providing the plurality of products to a feeder, separating the plurality of products, placing each of the separated products into a desired orientation, individually wrapping each of the separated products, to form a plurality of wrapped products, collating a number of the wrapped products into a group of products, storing a second number of the groups of products in a buffer, and placing two of the groups of products in the package. During the placing step, a divider is inserted between the groups of products.

### BRIEF DESCRIPTION OF THE DRAWINGS

FIG. 1 shows a top view of a tampon package according to the prior art;

FIG. 2 shows a top view of a tampon package according to the present disclosure;

FIG. 3 shows a front view of the divider of the present disclosure;

FIG. 4 shows a block diagram of the package manufacturing process of the present disclosure;

FIG. 5 shows a diagram of the collating step of the process shown in FIG. 4;

FIG. 6 shows a diagram of the buffer step of the process shown in FIG. 4;

FIG. 7 shows a second diagram of the buffer step of the process shown in FIG. 4; and

FIG. 8 shows a diagram of the cartoner step of the process shown in FIG. 4.

### DETAILED DESCRIPTION OF THE DISCLOSURE

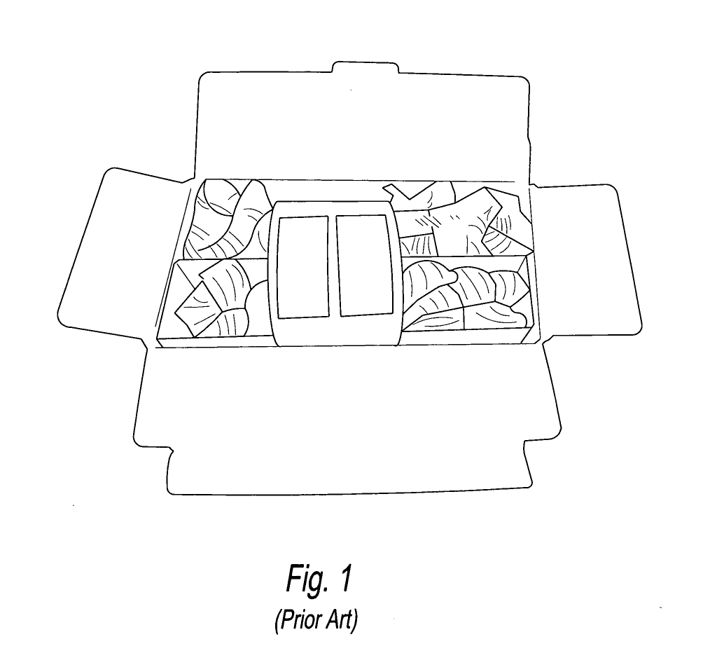

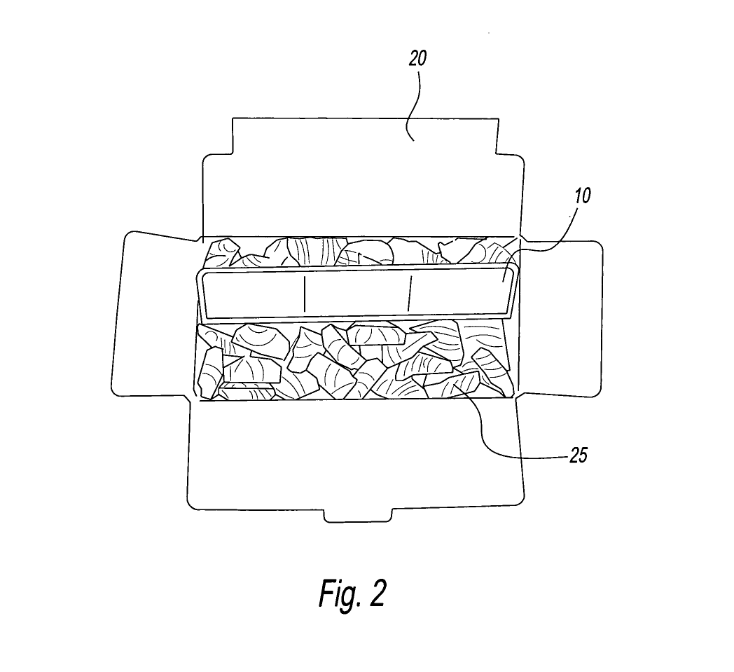

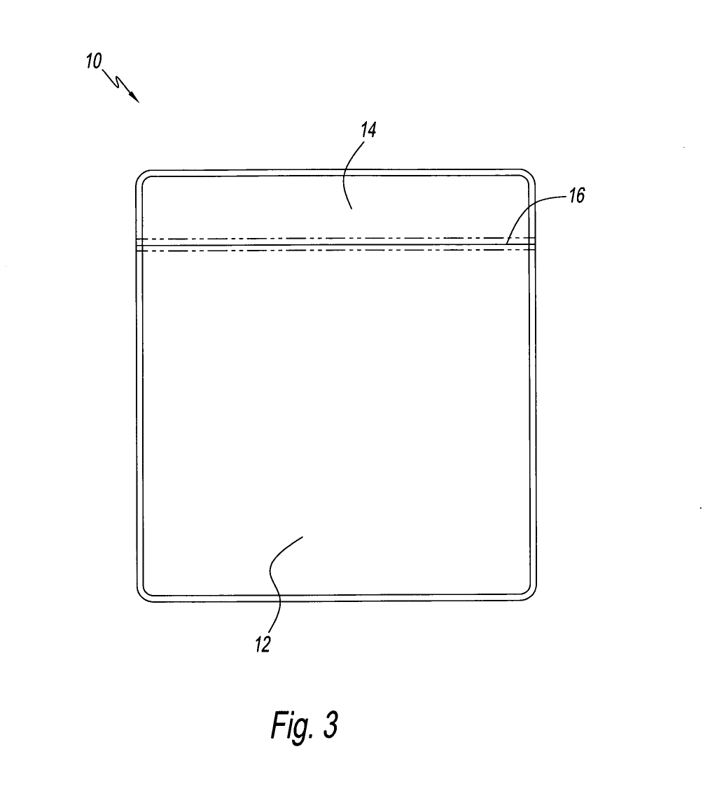

Referring to the drawings FIG. 1, there is shown a conventional tampon package. Referring to FIGS. 2 and 3, the present disclosure provides a divider 10 that can be included in a package 20 of tampons 25, during filling of package 20 with tampons 25. Divider 10 has a body 12 and a flag 14, which is connected to body 12 with perforated edge 16. Any information about the products, such as tampons 25, within package 20, in this case tampons 25, can be printed on flag 14 and/or body 12 of divider 10. Flag 14 can bend over the tampons 25 in package 20, which satisfies the FDA requirement that the warning information be prominently displayed when package 20 is opened. As will be discussed in greater detail below, the use of divider 10 eliminates a significant amount of the misfeeds that occur with currently used paper inserts, and therefore allows for much higher processing speeds and productivity.

In addition, divider 10 could serve a secondary but important additional role. Divider 10 can also be used in “multipacks”. These are packages 20 of tampons 25 having different absorbencies (e.g. regular, super etc.). Previously, when packing multipacks, tampons of one absorbency were grouped and placed into a sleeve. Another group of tampons having a different absorbency would then be added to the sleeved group, and the two groups would be placed in a package together. The paper insert would then be placed over the two groups of tampons. (See FIG. 1) By contrast, with divider 10, there would be no need for such a complex packaging operation. Divider 10 would not only convey useful safety and recommended usage information, but also divide tampons 25 into groups. Thus, when used in a multipack, divider 10 not only provides the above-discussed benefits over paper inserts, but also reduces the costs and processing steps associated with packaging tampons 25 in multipacks.

Divider 10 can be made of the same material as the stock that is used to make package 20, which can be 20-point board. In different embodiments, divider 10 can be made of 12-point board, or 10-point board. The present disclosure, however, contemplates other types of materials for divider 10, such as other weights and types of cardboard, or paper.

In traditional packaging methods, such as that used for the package shown in FIG. 1, the warning pamphlet or insert obstructs access to some of the tampons. This obstruction is an FDA requirement to ensure the consumer sees the warning information printed on the insert (See FIG. 1). When the tampons are packaged, a plurality of individually wrapped tampons are presented to a cartoner. Just before this step, the insert is placed across and perpendicular to the opening of the carton. Then the tampons are pushed along, so that the insert forms a “c” fold across the tampons, and the tampons and “c”-folded pamphlet are pushed into the carton. As previously discussed, this literature insertion process can be a constant source of machine downtime, as the paper insert often causes machine jams. When a multipack is produced, this adds a further level of complexity to the process, since the tampons, a separator, and the insert all have to be packaged together.

By contrast, in the high speed process of the present disclosure, divider 10 is used. As previously discussed, divider 10 can have the required warning information printed thereon. Divider 10 also has perforated edge 16, so that flag 14 can bend. This satisfies the requirement that the consumer see the warning information upon opening package 20. The consumer can also tear off flag 14, and still keep the remainder of divider 10 in place to keep different tampons separate, which is helpful when package 20 is a multipack. Although process 100 is described below in a tampon packaging application, process 100 can be useful in any packaging application where it is desirable to separate and/or provide information relating to the packaged products on a divider.

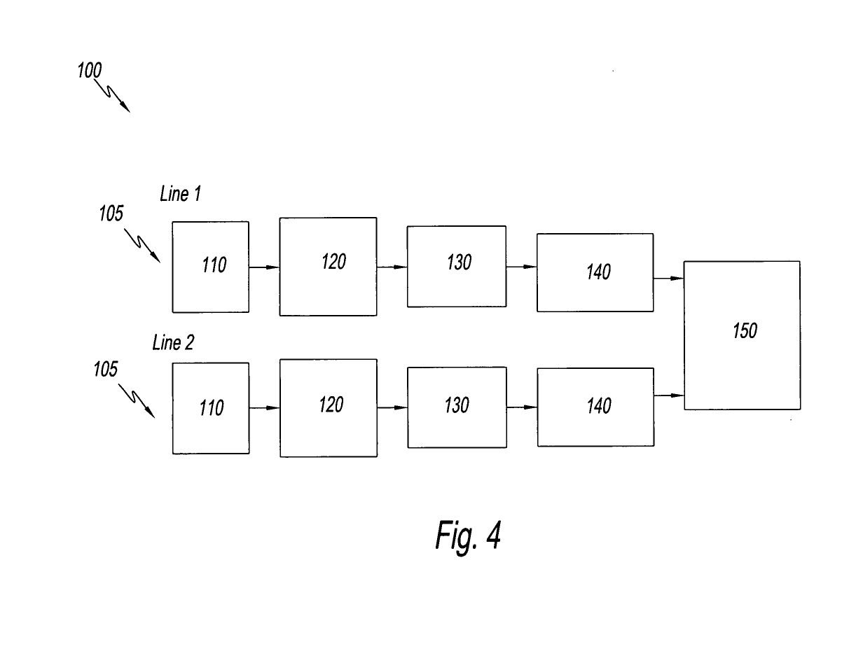

Referring now to FIGS. 4-8, a high-speed packaging method 100 according to the present disclosure is shown. The wrapping of tampons 25, and the placing of them into package 20 with divider 10, is a five step process, that has been found to greatly improve packaging speed. Method 100, represented in FIG. 4 and discussed in greater detail below, comprises a feeder 110 for separating and orientating the tampons 25 to be packaged, a wrapper 120 to wrap the individual tampons 25, a collator 130 for separating tampons 25 into groups 26, a buffer 140 for storing a number of groups 26 of tampons 25 leaving the collator, and a cartoner 150 for packaging groups 26 of tampons 25 into package 20. As shown in FIG. 4, there can be two separate lines 105 for feeding tampons 25 to cartoner 150. When method 100 is used to process a multipack package 20, each of lines 105 can process a different type of tampon 25.

Feeder 110 separates and orientates tampons 25 in a desired direction. After is leaving feeder 110, tampons 25 are transferred to wrapper 120, where they are wrapped individually. Tampons 25 are then transferred to collator 130.

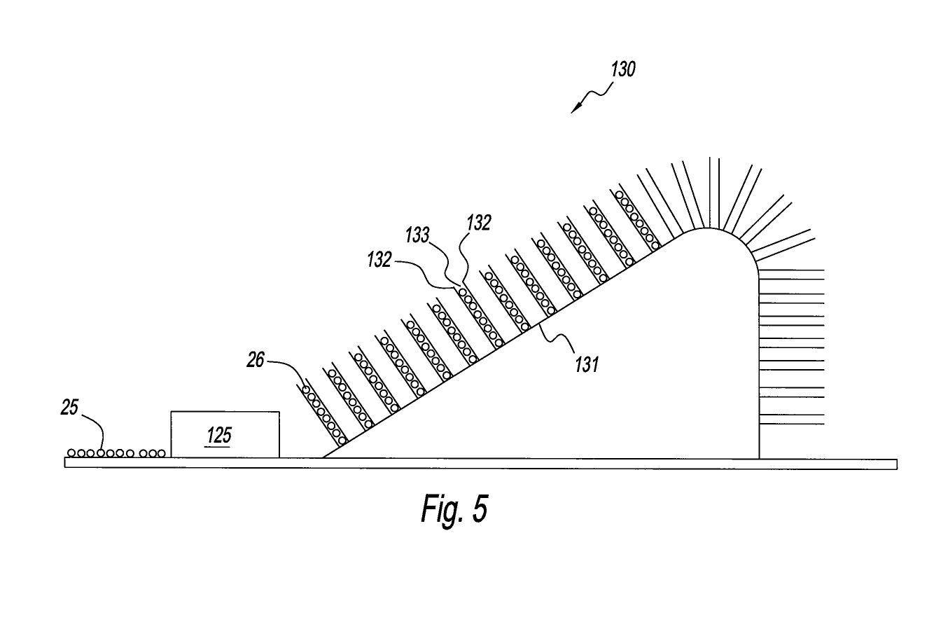

Referring to FIG. 5, collator 130 collates tampons 25 into a group 26. The amount of tampons 25 in group 26 will depend on the desired number of total tampons 25 for package 20. A counting unit 125 just prior to collator 130 counts tampons 25 into group 26, as they leave wrapper 120, and delivers them to incline paddle conveyer 131, which holds group 26 between two paddles 132. This assures that there is no gap or missing tampon 25. Two adjacent paddles 132 define a paddle area 133 where the group 26 of tampons 25 is kept, oriented in a single file row. When one paddle area 133 is filled, conveyer 131 advances to the next empty paddle area 133. As shown in FIG. 5, a pair of adjacent paddles 132 remain equidistant from each other, so that each paddle area 133 retains the same thickness as it travels along paddle conveyor 131.

This feature is highly advantageous, since it is much easier to process tampons 25 of group 26 when they are aligned in single file. In previously available collators employing conveyors and paddles, the products being collated were allowed to loosely fill the space between paddles. As the conveyor went around a bend, the space between adjacent paddles would grow, since the paddles would form a “v” shape. The products disposed in the space between the paddles would be loosely packed, and disorganized. When packaging tampons, this can be highly disadvantageous, since it makes processing the tampons more difficult and time-consuming. Collator 130 thus overcomes these drawbacks.

Two paddle conveyors 131 can be used in method 100, corresponding to the number of lines 105. Each paddle conveyor 131 can carry the same type of tampon 25, or in the case where package 20 is a multipack, each paddle conveyor 131 can carry a different type of tampon 25. Each paddle conveyor 131 can be programmed for a different number or group 26 of tampons 25, if desired. Each of groups 26 in each of paddle areas 133 are then transferred to buffer 140.

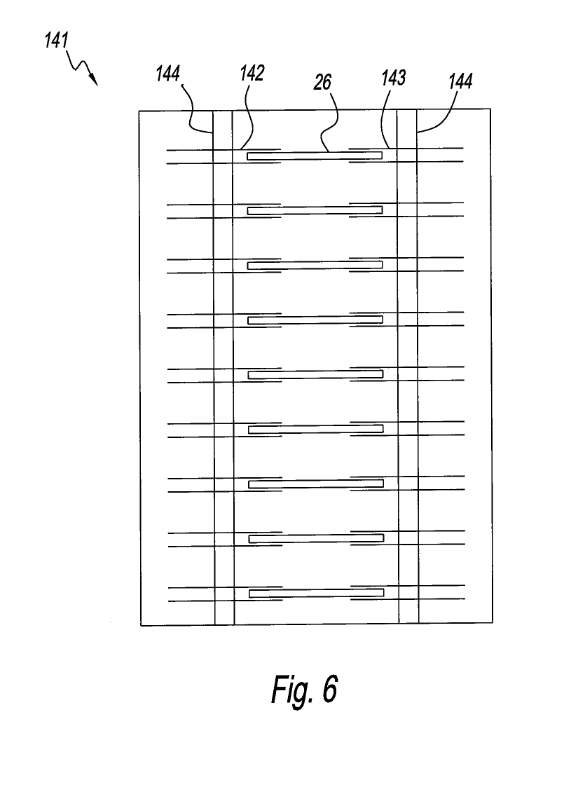

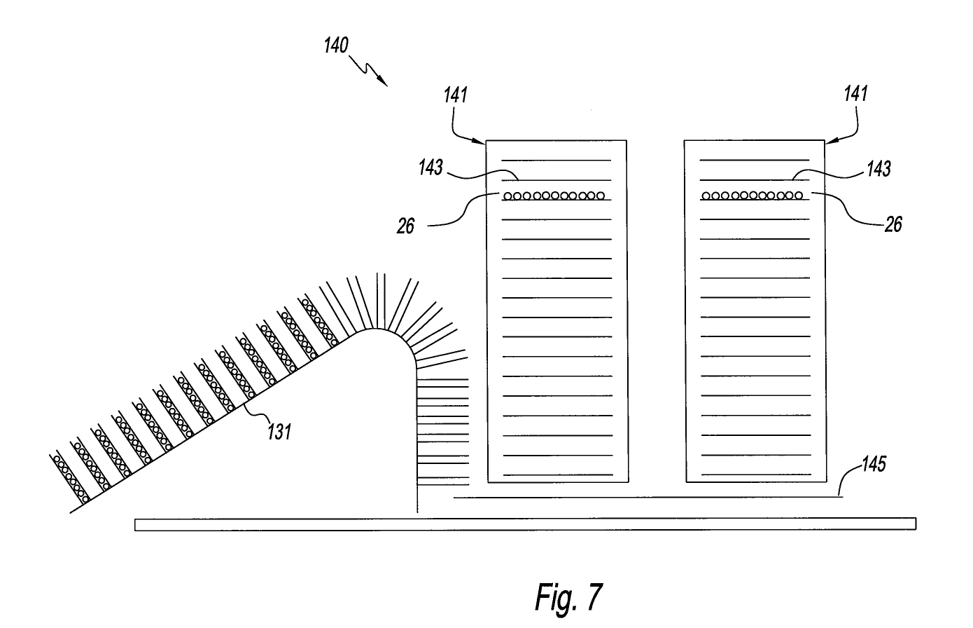

Referring to FIGS. 6-7, buffer 140 provides a time synchronization for each of lines 105. Buffer 140 comprises a number of towers 141, which store groups 26 of tampons 25 (after they are transferred from collator 130) between a series of left paddles 142 and right paddles 143. Paddles 142 and 143 are attached to belts 144 that can move up or down towers 141. A group 26 rides in gaps 145 between synchronized left paddles 142 and right paddles 143 on each tower. Belts 144 lift the groups 26, leaving a portion of the middle area of the tampon 25 open/free. When one tower 141 is filled, a stripper conveyor 145 can move groups 26 of tampons 25 to subsequent towers 141, by using a device (not shown) that can grab tampons 25 in the exposed area.

Thus, buffer 140 can carry enough groups 26 of tampons 25 to keep lines 105 running in the event that any or all of them shut down (e.g., if there is a machine jam or a manufacturing defect that causes a shutdown). In one embodiment, there are two towers 141 for each of lines 105, but the present disclosure contemplates any number of towers 141 per line 105. In one embodiment, buffer 140 can store enough groups 26 of tampons 25 to run the machine for four minutes, even when each or both of lines 105 are stopped.

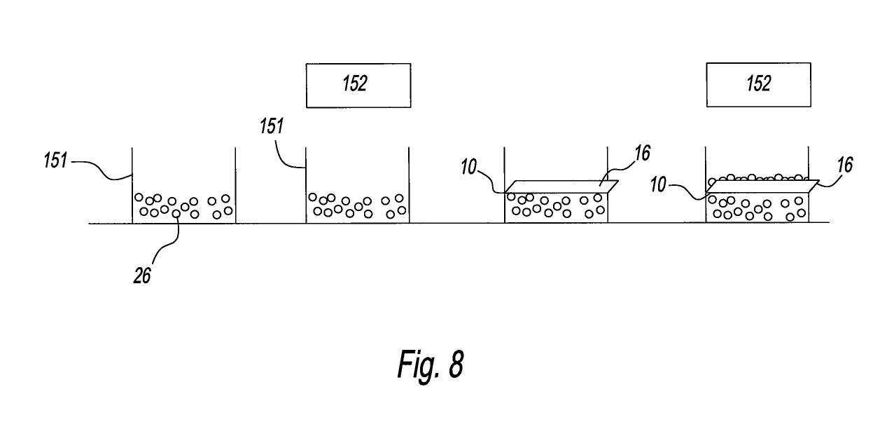

Referring to FIG. 8, after leaving buffer 140, groups 26 of tampons 25 are fed to cartoner 150. Cartoner 150 places groups 26, which can be of different sizes or numbers, into package 20, with divider 10 between the groups 26. The filling of package 20 starts when one of towers 141 is stripped of a first group 26, and that group 26 is delivered to a container 151. A leveling arm 152 then treats, or tamps down group 26 within container 151. Divider 10 is then placed on top of this first group 26. After the first group 26 of tampons 25 are delivered, and divider 10 is in place, a second group 26 of tampons 25 are added to container 151. Again, the tampons 25 are treated, or tamped down by leveling arm 152. An open package 20 is presented to container 151, first and second groups 26 of tampons 25, with divider 10 between them, are pushed into package 20. Flag 16 bends to one side, thus satisfying the requirement that the necessary warning information be shown upon the opening of package 20. The flaps of package 20 are then folded over and sealed.

The present disclosure having been thus described with particular reference to the preferred forms thereof, it will be obvious that various changes and modifications may be made therein without departing from the spirit and scope of the present invention as defined in the appended claims.

What is claimed is:

1. A package having a package body having an opposable top, a bottom, and a wall, to define an interior space, said interior space for receipt of a plurality of products, and a divider in said interior space, wherein said divider separates said products into at least two separate groups of products, and wherein said divider has information relating to said products printed thereon, said information being displayed prominently when the package body is opened.

2. The package of claim 1, wherein said divider has a body and a flag, and wherein said flag is connected to said divider body with a perforated edge.

3. The package of claim 2, wherein said information is on at least one of said divider body and said flag.

4. The package of claim 2, wherein said flag bends over one of said at least two groups of products, so that a view of said plurality of products is at least partially obstructed when said package is open, for prominent viewing of said flag.

5. The package of claim 2, wherein said flag is detachably connected to said divider body.

6. The package of claim 1, wherein said products are tampons.

7. A method of filling a package with a plurality of products, comprising:
providing the plurality of products to a feeder;
separating the plurality of products;
placing each of the separated products into a desired orientation;
individually wrapping each of said separated products, to form a plurality of wrapped products;
collating a number of said wrapped products into a group of products;
storing a second number of said groups of products in a buffer; and
placing two of said groups of products in said package,
wherein during said placing step, a divider is inserted between said groups of products.

8. The method of claim 7, wherein during said collating step, each of said groups of products are conveyed to said buffer with a conveyor, said conveyor having a plurality of pairs of paddles thereon, wherein a distance between the two paddles in each of said pairs of paddles remains constant as said conveyor conveys said groups of products to said buffer.

9. The method of claim 7, wherein between said storing and said placing step, the method further comprises:
placing a first of said groups of products in a container;
placing said divider over said first group of products; and
placing a second of said groups of products over said divider.

10. The method of claim 9, wherein said each of said first and second groups of products are leveled after being placed in said container.
